(19) United States
(12) Patent Application Publication
(10) Pub. No.: US 2005/0256206 A1
(43) Pub. Date: Nov. 17, 2005

(54) Enantiomers of O-desmethyl venlafaxine

(75) Inventors: John P. Yardley, King of Prussia, PA (US); Andre A. Asselin, Mahwah, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/183,573

(22) Filed: Jul. 18, 2005

Related U.S. Application Data
(63) Continuation of application No. 10/799,321, filed on Mar. 12, 2004, now abandoned.
Application No. 10/799,321 is a continuation of application No. 10/373,145, filed on Feb. 24, 2003, now abandoned.
Application No. 10/373,145 is a continuation of application No. 10/154,994, filed on May 23, 2002, now abandoned.
Application No. 10/154,994 is a continuation of application No. 09/957,908, filed on Sep. 21, 2001, now abandoned.
Application No. 09/957,908 is a continuation of application No. 09/590,741, filed on Jun. 8, 2000, now abandoned.
(60) Provisional application No. 60/183,029, filed on Jun. 15, 1999.

(51) Int. Cl. A61K 31/137

(52) U.S. Cl. 514/650; 564/339

(57) ABSTRACT

This invention provides pharmaceutically active enantiomers of the venlafaxine metabolite O-Desmethyl venlafaxine, R(−)-4-[2-(Dimethylamino-1-(1-hydroxycyclo-hexyl)ethyl]phenol or R(−)1-[2-(dimethylamino)-1-(4-hydroxyphenyl)ethyl]cyclo-hexanol, and S(+)-1-[2-(Dimethylamino)-1-(4-hydroxyphenyl)ethyl]cyclohexanol or S(+)-4-[2-(Dimethylamino)-1-(1-hydroxycyclohexyl)ethyl]phenol, or one or more pharmaceutically acceptable salts or salt hydrates thereof, as well as pharmaceutical compositions utilizing these enantiomers and methods of using the enantiomers to treat, inhibit or control central nervous system disorders.

This application claims the benefit of U.S. Provisional Application No. 60/183,029, which was converted from U.S. patent application Ser. No. 09/333,594, filed Jun. 15, 1999, pursuant to a petition filed under 37 C.F.R. 1.53(c)(2)(i).

This invention provides enantiomers of O-desmethyl venlafaxine, (R/S) 4-[2-(Dimethylamino-1-(1-hydroxycyclohexyl)ethyl]phenol, as well as pharmaceutical compositions and uses thereof.

### BACKGROUND OF THE INVENTION

Various patents and literature references describe the biological activities of venlafaxine, and its salts and analogs. Venlafaxine hydrochloride tablets are marketed by Wyeth-Ayerst Laboratories under the Effexor® trademark.

The absolute configuration of the (+) enantiomer of venlafaxine was established as S by a single crystal X-ray analysis of the hydrobromide salt and the anomalous dispersion technique (Yardley et al., J. Med. Chem., 1990, 33, 2899).

(R/S)-1-[2-(dimethylamino)-1-(4-methoxyphenyl)ethyl]cyclohexanol and its metabolites 1-[2-(dimethylamino)-1-(4-hydroxyphenyl)ethyl]cyclohexanol and 1-[1-(4-methoxyphenyl)-2-(methylamino)ethyl]cyclohexanol are disclosed and claimed in U.S. Pat. No. 4,535,186 (Husbands et al.). U.S. Pat. No. 5,530,013 (Husbands et al.) claims the use of venlafaxine in the inducement of cognition enhancement. U.S. Pat. No. 5,506,270 (Upton et al.) claims venlafaxine's use in methods of treating hypothalamic amenorrhea in non-depressed women.

U.S. Pat. No. 5,788,986 (Dodman) and U.S. Pat. No. 5,554,383 (Dodman) teaches and claims the use of serotonin reuptake inhibitors in modifying the behavior of dogs.

### SUMMARY OF THE INVENTION

This invention provides pharmaceutically active enantiomers of the venlafaxine metabolite O-Desmethyl venlafaxine, R(−)-4-[2-(Dimethylamino-1-(1-hydroxycyclo-hexyl)ethyl]phenol and S(+)₄-[2-(Dimethylamino)-1-(1-hydroxycyclo-hexyl)ethyl]-phenol, or a pharmaceutically acceptable salt or salt hydrate thereof, having the structures:

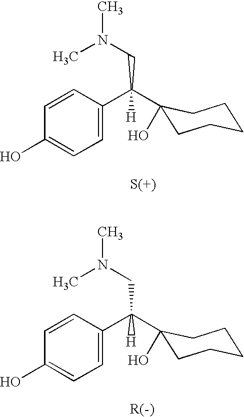

Particularly, this invention provides compositions of matter of both the R(−) enantiomer and S(+) enantiomer substantially free of each other. Under a different system of nomenclature S(+)-4-[2-(Dimethylamino) 1-(1-hydroxycyclohexyl)-ethyl]phenol may also be named S(+)-1-[2-(Dimethylamino-1-(4-hydroxyphenyl)-ethyl]cyclohexanol. Similarly, R(−)₄-[2-(Dimethylamino-1-(1-hydroxycyclohexyl)-ethyl]phenol may also be referred to R(−)1-[2-(dimethylamino)-1-(4-hydroxyphenyl)-ethyl]cyclohexanol. As used herein, the designations (+) and (−) refer to the sign of rotation of the relevant free base.

These enantiomers and their pharmaceutically useful salts and hydrates are useful for the biological and pharmacological activities for which venlafaxine and its salts are known in the art. These enantiomers may be used in treating or inhibiting central nervous system disorders, including depression, panic disorder, post-traumatic stress disorder, late luteal phase dysphoric disorder (also known as pre-menstrual syndrome), attention deficit disorder, with and without hyperactivity, generalized anxiety disorder, bulimia nervosa, Gilles de la Tourette Syndrome, Shy Drager Syndrome, vasomotor flushing, drug and alcohol addiction, sexual dsifunction (including premature ejaculation), borderline personality disorder, chronic fatique syndrome, fibromyalgia, urinary incontinence and others. These compounds are also useful in the inducement of cognition enhancement and in regimens for cessation of smoking or other tobacco uses.

Racemic 1-[2-(dimethylamino)-1-(4-hydroxyphenyl)ethyl]cyclohexanol can be produced as described in Example 26 of U.S. Pat. No. 4,535,186 (Husbands et al.), which is incorporated herein by reference. It will be understood that the enantiomers may be separated from each other by standard resolution techniques known in the art.

Alternatively, these R and S enantiomers may be obtained by O-demethylation of the separated enantiomers of venlafaxine using either boron tribromide or ethane thiol anion.

### EXAMPLE NO. 1

### 1-[2-(Dimethylamino-1-(4-hydroxyphenyl)ethyl]cyclohexanol fumarate hydrate

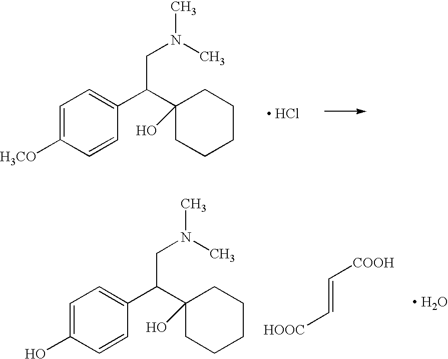

1-[2-(Dimethylamino-1-(4-methoxyphenyl)ethyl]cyclohexanol —HCl (200 g=0.6372 mol) was dissolved in H₂O (500 mL). CH₂Cl₂ (350 mL) was added thereto and this mixture cooled to 10° C. At this temperature 2.5 N NaOH (280 mL=0.7 mol) was added slowly over 1 hr. CH₂C₂ was separated and the aqueous layer extracted with CH₂CL, (200 mL). Combined CH₂Cl₂ extracts were dried (MgSO₄) filtered, and evaporated in vacuo to yield 1-[2-(Dimethylamino-1-(4-methoxyphenyl)-ethyl]cyclohexanol free base (167.5 g=94.5%) as white solid, mp 77-79° C.

To a stirred solution of 1-[2-(Dimethylamino-1-(4-methoxyphenyl)ethyl]-cyclohexanol-free base (13.87 kg=50 mmols) in CH₂Cl₂ (300 mL) cooled to 40° C., under N₂ was added slowly BBr₃ (10 mL=105.5 mmols) over a period of 15 minutes. The reaction mixture warmed to 0° C. where it stirred for 3 hrs. During this time a gummy precipitate formed. Still at 0° C., 2.5N NaOH (200 mL) was added slowly over 1 hr, then allowed to warm to room temperature and stirred for 3 hrs. CH₂Cl₂ was removed by evaporation under reduced pressure leaving an aqueous layer having a pH=13-14. Aqueous layer was extracted with EtOAc (3×100 mL) and its pH dropped to 9. Combined EtOAc extracts were dried (MgSO4); filtered, evaporated in vacuo to afford crude phenol (9.3 g=71%) as a white solid, mp 208-213° C. (TLC) together with some dehydrated product. This crude product was used in the next step without further purification.

Crude phenol (9.3 g=35.31 mmoles) and fumaric acid (4.91 g=42.37 mmoles) were dissolved in a mixture of methanol/acetone (1:3) (195 mL), stirred at room temperature for 15 minutes. H₂O (0.8 mL=44.44 mmoles) was added to the clear light yellow solution. The whole was stirred at room temperature for 3 hours. The resulting white precipitate was filtered, washed with acetone (30 mL) dried in air to yield 8.0 g=57% white solid, mp: 145-150° C.

### EXAMPLE NO. 2

### R(−)-1-[2-(Dimethylamino-1-(4-methoxyphenyl)ethyl]cyclohexanol

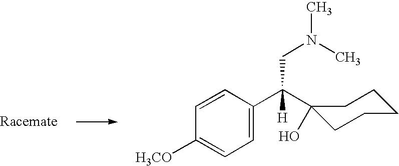

To a solution of yield 1-[2-(Dimethylamino-1-(4-methoxyphenyl)ethyl]-cyclohexanol free base (100 g=0.36 mol) in EtOAc (750 mL) at room temp was added at once a solution of (+)-Di-para Toluoyl-D-tartaric acid-monohydrate (DT(−)T; 40 g=0.0991 mol]. The whole was stirred at room temp for 1 hr. The resulting precipitate was filtered off, washed with EtOAc (3×100 mL), dried overnight at 35° C. in a vacuum oven to provide crude R(−)-1-[2-(dimethylamino)-1-(4-methoxyphenyl)-ethyl]cyclohexanol DT(−)T salt (83 g=92.8%) as a white solid.

### Recrystallization of R(−) 1-[2-(Dimethylamino-1-(4-methoxyphenyl)-ethyl]cyclohexanol, DT(−)T Salt

Crude DT(−)T salt (83 g) was dissolved in EtOAc (700 mL). The mixture was heated up to reflux. Methanol (75 mL) was added thereto to obtain a clear solution. The mixture was concentrated at atmospheric pressure to a volume of 400 mL (some solid started to precipitate). The mixture was cooled at 25° C. for 1 hr, then at 0° C. for another 2 hrs and filtered off to provide (−)1-[2-(Dimethylamino-1-(4-methoxyphenyl)ethyl]cyclohexanol DT(−)T salt (63 g=77%). NOTE: Optical rotation of this salt in ethanol was +47.0°.

### Isolation of R(−)1-[2-(Dimethylamino-1-(4-methoxyphenyl)-ethyl]cyclohexanol Base

R(−)1-[2-(Dimethylamino-1-(4-methoxyphenyl)ethyl]cyclohexanol DT(−)T salt was slurried in a mixture of H₂O/CH₂Cl₂ (400 mL/400 mL). The pH of this mixture was adjusted to 13 by adding 25% NaOH solution (120 mL). The layers were separated, aqueous layer was extracted with CH₂Cl₂ (1×200 mL). Combined CH₂Cl₂ layers were washed with H₂O (2×200 mL) saturated NaCl solution (1×200 mL), dried (MgSO₄) and evaporated at atmospheric pressure to a volume of 100 mL. Hexane (300 mL) was added thereto; and solution became hazy. After charcoal treatment (1 teaspoon), the filtrate was concentrated at atmospheric pressure to a volume of 250 mL and allowed to cool. The resulting white precipitate was collected by filtration, washed well with hexane (2×100 mL), dried in a vacuum oven overnight to provide R(−)1-[2-(Dimethylamino-1-(4-methoxyphenyl)ethyl]cyclohexanol base (28.5 g). Recrystallization from CH₂/Cl₂/hexane (50 mL/200 mL) gave analytically pure R(−)1-[2-(Dimethylamino-1-(4-methoxyphenyl)ethyl]cyclohexanol base (23.5 g=23.5%) Rotation=−31.08° (in ethanol). Anal. Calcd: C, 73.60; H, 9.81; N, 5.05 Found: C, 73.75, H, 9.73; N, 4.83.

### EXAMPLE NO. 3

### R(−)-1-[2-(Dimethylamino-1-(4-hydroxyphenyl)ethyl]cyclohexanol fumarate hydrate salt

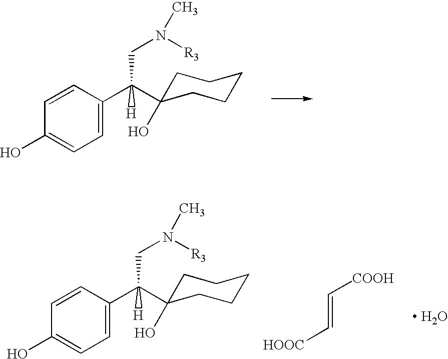

To a cooled (−40° C.) stirred solution of R(−)-1-[2-(dimethylamino)-1-(4-methoxyphenyl)ethyl]cyclohexanol (13.87 g=50 mmol) in CH₂Cl₂ (500 mL) under nitrogen was added slowly BBr₃ (10 mL=105.5 mmols) over a period of 15 min. The reaction mixture warmed to 0° C. where it stirred for 3 h. During this time a gummy precipitate formed. Still at 0° C., 2.5 N NaOH solution (200 mL) was added slowly over 1 hr. The reaction mixture was allowed to warm to room temp and stirred overnight. Methylenechloride was removed, leaving an aqueous layer having a pH-13-14. Aqeuous layer was extracted with EtOAc (3×100 mL) and its pH dropped in 9. Combined EtOAc extracts were washed with brine, dried (MgSO₄) and evaporated in vacuo to give crude phenol 6.5 g-49.4%) as white solid. The crude phenol (6.5 g=24.68 mmols) and fumaric acid (1.2 eq; 3.3 g=29.61 mmols) were dissolved in a mixture of methanol/acetone (1:3) (135 mL) and stirred at room temp for 15 min. After this time H₂O (0.6 mL) was added to the clear light yellow colored solution. Precipitation was seen immediately. The suspension was stirred at room temp for 3 h. The resulting white precipitate was collected by filtration, washed well with acetone (1×35 mL) and dried to provide title compound (6.6 g=67.3%, mp 150-155° C. This was recrystallized from MeOH/Acetone/H₂O (40 mL: 126 mL: 0.3 mL) to give 3.7 g (37.7%) of analytically pure R(−)-4-[2-(Dimethylamino)-1-(1-hydroxycyclo-hexyl)ethyl]phenol fumarate hydrate salt. Rotation: +6.60° (in methanol) for the fumarate hydrate, −15.58° (in methanol) for the free base. Anal. Calcd: C, 60.43; H, 7.86; N, 3.52. Found: C, 60.16; H, 7.64; N, 3.36.

### EXAMPLE NO. 4

### R(−)-4-[2-(Dimethylamino)-1-(1-hydroxycyclohexyl)ethyl]phenol fumarate hydrate salt

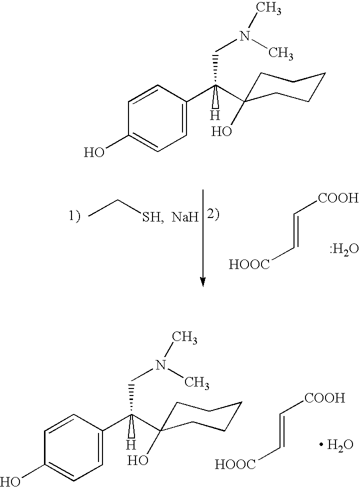

Under gentle N₂ stream 60% NaH (35 mmols=1.4 g) was washed with hexane, collected by filtration and transferred into a 250 mL 3 neck flask. DMF (20 mL) was added into the flask to cover the sodium hydride and the suspension cooled to 10° C. Under stirring a solution of ethane thiol (32.40 mmols=2.075 g=2.47 mL) in DMF (5 mL) was added dropwise over 40 min. During the addition of the ethanethiol solution, foaming was noted in the flask. Stirring was continued at 20° C. for 1½ h and the starting material R(−)-1-[2-(dimethylamino)-1-(4-methoxyphenyl)ethyl] cyclohexanol (12.5 mmols=3.46 g) was added as a solid over 5 min. The reaction mixture was heated up slowly to 150° C. over 30 min. and stirred at this temperature for another hour. After this time the yellow brownish colored reaction mixture was rapidly cooled to 25° C. and quenched by adding it into a flask containing H₂O (90 mL). The mixture was charcoaled and filtered through celite. The mixture was washed with H₂O (1×25 mL) and 1N NaOH (1×50 mL). At this point the p H of the clear yellow colored solution was 13. This was extracted with toluene (1×60 mL), followed by hexane (1×60 mL). Under stirring at room temp it was neutralized to pH=9 with conc. HCl (3 mL). The resulting suspension was cooled at 5° C., stirred for 1 h and the white solid was collected by filtration, dried in air overnight to give 2.6 g=79% of the phenol (mp: 232-235° C.). This compound (9.491 mmols=2.5 g) and fumaric acid (11.39 mmols=1.32 g) were dissolved in hot methanol/acetone (1:3) mixture (54 mL) and filtered leaving a clear light yellow colored solution. Under stirring at room temp H₂O (0.227 mL) was added to the solution. The solution became cloudy immediately. Stirring was resumed for 2 h and the resulting white solid was collected by filtration, washed with acetone (2×50 mL), dried at 35° C. in a vacuum oven overnight to give 3 g=60% of analytically pure compound.

### EXAMPLE NO. 5

### S(+)-1-[2-(Dimethylamino)-1-(4-hydroxyphenyl)ethyl]cyclohexanol fumarate hydrate salt

### a) S(+)-1-[2-(Dimethylamino)-1-(4-methoxyphenyl)ethyl]cyclohexanol

To the mother liquor from the resolution after separation of R(−)-1-[2-(dimethylamino)-1-(4-methoxyphenyl)ethyl]cyclohexanol DT(−)T salt (see Example No. 2) was added H₂O (400 mL). The mixture has a pH=7. The pH was adjusted to 12 by adding 25% NaOH solution (150 mL). EtOAc layer was separated, washed with saturated NaCl solution (2×100 ml) dried (MgSO₄) and concentrated in vacuo to a volume of 100 mL. Hexane (400 mL) was added thereto and the whole was stirred at room temp for 1 h, then at 0° C. for another 2 h. The white precipitate was collected by filtration to give 37.5 g. This was dissolved in hot CH₂Cl₂ (110 mL). Charcoal (2 g) was added to the hot solution and stirred for 5 minutes. After filtration through solka floc, hexane (380 mL) was added to the filtrate. The mixture was concentrated at atmospheric pressure to a volume of 250 mL and allowed to stay at room temp overnight. The resulting white precipitate was collected by filtration to provide 29.7 g. Recrystallization from CH₂Cl₂/Hexane (72/249 mL) gave analytically pure S(+)-1-[2-(dimethylamino)-1-(4-methoxyphenyl)ethyl]-cyclohexanol, 25.4 g=25.4% yield. Rotation: +28.82° (in ethanol.). Anal. Calcd.: C, 73.60; H, 9.81; N, 5.05. Found: C, 73.70; H, 10.10; N, 4.85.

### b) S(+)-1-[2-(Dimethylamino)-1-(4-hydroxyphenyl)ethyl]cyclohexanol fumarate hydrate salt

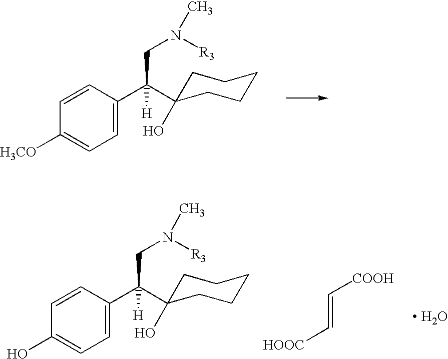

To a stirred solution of S(+)-1-[2-(dimethylamino)-1-(4-methoxyphenyl)ethyl]-cyclohexanol (13.87 g=50 mmols) in CH₂Cl₂ (500 mL), cooled to −40° C. under nitrogen was added slowly BBr₃ (10 mL=105.5 mmols) over a period of 15 min. The reaction mixture warmed to 0° C. where it was stirred for 3 h. During this time a gummy precipitate formed. Still at 0° C., 2.5 N NaOH (200 mL) was added slowly over 1 h. Then the mixture was allowed to warm to room temp and stirred overnight. CH₂Cl₂ was removed under vacuo leaving an aqueous layer having a pH=13-14. Aqueous layer was extracted with EtOAc (3×100 mL) and its pH dropped to 9. Combined EtOAc extracts were washed with saturated NaCl solution, dried (MgSO₄), filtered and evaporated in vacuo to afford crude phenol (3.9 g=29.6%) as a white solid. Crude phenol (3.9 g=14.8 mmols) and fumaric acid (2.06 g=17.77 mmols) were dissolved in a mixture of methanol/acetone (1:3) (81 mL) and stirred at room temp for 15 min. H₂O (0.325 mL=18 mmol) was added to the clear light yellow solution. Precipitation was noted immediately. The whole was stirred at room temp for 3 h. The resulting white precipitate was collected by filtration, washed with acetone (1×35 mL) and dried to give 2.4 g (40.8%) of product. Recrystallization from MeOH/Acetone/H₂O (14 mL/46 mL/0.325 mL) gave 2.1 g=35% of analytically pure S(+)-4-[2-(Dimethylamino)-1-(1-hydroxycyclohexyl)ethyl]phenol fumarate hydrate salt. Rotation: −6.56° (in methanol) for the fumarate hydrate. +9.07° (in methanol) for the free base. Anal. Calcd: C, 60.43; H, 7.86; N, 3.52. Found: C, 60.47; H, 7.51; N, 3.32.

### EXAMPLE NO. 6

### S(+)-4-[2-(Dimethylamino)-1-(1-hydroxycyclohexyl)ethyl]phenol fumarate hydrate salt

Under gentle N₂ stream 60% NaH (35 mmols=1.4 g) was washed with hexane, collected by filtration and transferred into a 250 mL 3 neck flask. DMF (20 mL) was added into the flask to cover the sodium hydride and the suspension cooled to 10° C. Under stirring a solution of ethanthiol (32.40 mols=2.075 g=2.47 mL) in DMF (5 mL) was added dropwise over 40 min. During the addition of the ethanthiol solution, foaming was noted in flask. Stirring was continued at 20° C. for 1½ h and the starting material S(+)-1-[2-(dimethylamino)-1-(4-methoxyphenyl)ethyl]-cyclohexanol (12.5 mmols=3.46 g) was added as a solid over 5 min. The reaction mixture was heated up slowly to 150° C. over 30 min and stirred at this temperature for another hour. After this time the yellow brownish colored reaction mixture was rapidly cooled to 25° C. and quenched by adding it into a flask containing H₂O (90 mL). The mixture was charcoaled and filtered through celite. The cake was washed with H₂O (1×25 mL) and 1N NaOH (1×50 mL). At this point the pH of the clear yellow colored solution was 13. This was extracted with toluene (1×60 mL), followed by hexane (1×60 mL). Under stirring at room temperature it was neutralized to pH=9 with conc. HCl (3 mL). The resulting suspension was cooled at 5° C., stirred for 1 h and the white solid was collected by filtration, dried in air overnight to give 2.4 g=72% of the phenol (mp 230-232° C.). This compound (8.35 mmols=2.2 g) and fumaric acid (10.023 mmols=1.163 g) were dissolved in hot methanol/acetone (1:3) mixture (48 mL) and filtered leaving a clear light yellow colored solution. Under stirring at room temperature H₂O (0.2 mL) was added to the solution. The solution became cloudy immediately. Stirring was resumed for 2 h and the resulting white solid was collected by filtration, washed with acetone (2×50 mL) and dried at 35° C. in a vacuum oven overnight to give 2.4 g of analytically pure compound.

Tests were conducted to examine the effects of these compounds at 5-HT2 receptor sites and on monamine uptake.

### Methods

Male Sprague-Dawley rats (180-260 g, Charles River) were used in all neurochemical assays. Rats were housed in temperature-controlled quarters on a 12 hr light/12 hr dark cycle with free access to food and water.

Neurotransmitter Uptake Inhibition

Uptake experiments were performed using a crude synaptosomal preparation made from the brain tissue of adult male Sprague-Dawley rats. The cortex of 1 rat for NE and 5-HT uptake was removed on ice and homogenized in 20 volumes of 0.32 M sucrose/g tissue weight using a Potter-Elvehjem teflon homogenizer (3 strokes at 840 rpm). The homogenate was then centrifuged for 12 minutes at 1,000×g at 0-4° C. The resulting supernatant was decanted into a chilled glass beaker and kept on ice until assayed. Protein concentration was determined by the method of Lowry et al. (1).

For these experiments, all compounds were run in duplicate in concentrations of 0.003-30.0 μM. Each tube received buffer (790 μl in drug tubes, 800 μl in control tubes), 10 μl of drug or standard (0.1 μM DMI for NE uptake and 3.0 μM zimelidine HCl for 5-HT uptake), 100 μl isotope (0.1 μM ³H—NE and 0.05 μM ¹⁴C-5-HT), and 100 μl tissue. Tubes were incubated at 37° C. for 6 minutes. Incubation was terminated by the addition of 2.5 ml buffer followed by vacuum filtration using a Brandel filtration manifold with Whatman GF/B glass fiber filters and a second wash with 2.5 ml buffer. Filters were added to 10 ml Hydrofluor, shaken for 15 minutes and counted in a Packard 460CD scintillation counter equipped with dual-label dpm data reduction. Results were expressed as pmol uptake/mg protein/min. IC₅₀'s for uptake inhibition were calculated by linear regression of logit [% of active uptake] vs. log [concentration of test drug].

Results

O-Desmethyl venlafaxine, 4-[1-(2-dimethylamino)-2-(1-hydroxycyclohexyl)-ethyl]-phenol, and its S(+) and R(−) enantiomers were tested for their ability to inhibit NE and 5-HT neurotransmitter uptake. O-Desmethyl venlafaxine inhibited 5-HT uptake (IC₅₀s=0.20 μM). Both enantiomers of O-Desmethyl venlafaxine were active in inhibiting 5-HT uptake with the (−) enantiomer being the more potent [(+)O-Desmethyl venlafaxine IC₅₀=0.12 μM; (−)O-Desmethyl venlafaxine=0.06 μM]. Venlafaxine and O-Desmethyl venlafaxine also inhibited NE uptake (IC₅₀=0.72 μM and 75% inhibition at 0.3 μM, respectively). Both enantiomers of O-Desmethyl venlafaxine also inhibited NE uptake [(+)O-Desmethyl venlafaxine IC₅₀=0.72 μM; (−) O-Desmethyl venlafaxine IC₅₀=0.27 μM]. The (−) enantiomer of O-Desmethyl venlafaxine was more potent in inhibiting NE uptake.

Pharmaceutical compositions and formulations containing the enantiomers described herein can be produced in the same fashion and containing the same dosages as those described in the art for venlafaxine hydrochloride. The pharmaceutical formulations or compositions of this invention include those having as an active ingredient the R(−) enantiomer of O-Desmethyl venlafaxine substantially free of S(+) O-Desmethyl venlafaxine. This invention also includes formulations in which an active ingredient is the S(+) enantiomer of O-Desmethyl venlafaxine substantially free of R(−) O-Desmethyl venlafaxine. Each of these formulations also comprises one or more pharmaceutically useful excipients, carriers or adjuvants.

Formulations of the present invention may be produced using the S or R enantiomer of O-Desmethyl venlafaxine, or a pharmaceutically acceptable salt or salt hydrate thereof, in the same fashion as described for venlafaxine formulations in U.S. Pat. No. 5,530,013 (Husbands et al.) and U.S. Pat. No. 5,506,270 (Upton et al.), both of which are incorporated herein by reference.

Preferred oral extended release formulations of this invention are comprised of the active enantiomer in admixture with microcrystalline cellulose and hydroxypropylmethylcellulose. Formed as beads or spheroids, the drug containing formulation is coated with a mixture of ethyl cellulose and hydroxypropylmethyl cellulose to provide the desired level of coating, generally from about two to about twelve percent on a weight/weight basis of final product or more preferably from about five to about ten percent (w/w), with best results obtained at from about 6 to about 8 percent (w/w). More specifically, the extended release spheroid formulations of this invention comprise from about 30 to 40 percent R(−) O-desmethyl venlafaxine, from about 50 to about 70 percent microcrystalline cellulose, NF, from about 0.25 to about 1 percent hydroxypropylmethylcellulose, USP, and from about 5 to about 10 percent film coating, all on a weight/weight basis. And preferably, the spheroid formulations contain about 35 percent active ingredient, about 55 to 60 percent microcrystalline cellulose NF (Avicel® PH101), about one half percent hydroxypropyl methylcellulose 2208 USP (K3, Dow, which has a viscosity of 3 cps for 2% aqueous solutions, a methoxy content of 19-24% and a hydroxypropoxy content of 4-13%), and from about 6 to 8 percent film coating.

The film coating is comprised of 80 to 90 percent of ethyl cellulose, NF and 10 to 20 percent hydroxypropyl methylcellulose (2910), USP on a weight/weight basis. Preferably the ethyl cellulose has a ethoxy content of 44.0-51% and a viscosity of 50 cps for a 5% aqueous solution and the hydroxypropylmethylcellulose is USP 2910 having a viscosity of 6 cps at 2% aqueous solution with a methoxy content of 28-30% and a hydroxypropoxy content of 7-12%. The ethyl cellulose used herein is Aqualon HG 2834.

Other equivalents of the hydroxypropylmethylcelluloses 2208 and 2910 USP and ethyl cellulose, NF, having the same chemical and physical characteristics as the proprietary products named above may be substituted in the formulation without changing the inventive concept. Important characteristics of suitable hydroxypropylmethylcelluloses include a low viscosity, preferably less than 10 cps and more preferably 2-5 cps, and a gel temperature above that of the temperature of the extrudate during extrusion. As explained below, these and other characteristics which enable the extrudate to remain moist and soft (pliable) are preferred for the hydroxypropylmethylcellulose. In the examples below, the extrudate temperature was generally 50-55° C.

Specific examples of extended release compositions of this invention include the following.

### FORMULATION EXAMPLE 1

A mixture of 44.8 parts (88.4% free base) of O-desmethyl venlafaxine or a salt or hydrate thereof, such as the fumarate hydrate salt, 74.6 parts of the microcrystalline cellulose, NF, and 0.60 parts of hydroxypropylmethyl cellulose 2208, USP, can be blended with the addition of 41.0 parts water. The plastic mass of material is then extruded, spheronized and dried to provide uncoated drug containing spheroids.

Stir 38.25 parts of ethyl cellulose, NF, HG2834 and 6.75 parts of hydroxypropyl methylcellulose 2910, USP in a 1:1 v/v mixture of methylene chloride and anhydrous methanol until solution of the film coating material is complete.

To a fluidized bed of the uncoated spheroids apply 0.667 parts of coating solution per part of uncoated spheroids to obtain extended release, film coated spheroids having a coating level of 3%.

The spheroids can then be sieved to retain the coated spheroids of a particle size between 0.85 mm to 1.76 mm diameter. These selected film coated spheroids are filled into hard gelatin capsules conventionally.

### FORMULATION EXAMPLE 2

Same as for Example 1 except that 1.11 parts of the film coating solution per part of uncoated spheroids is applied to obtain a coating level of 5%.

### FORMULATION EXAMPLE 3

Same as for Example 1 except that 1.33 parts of the film coating solution is applied to 1 part of uncoated spheroids to obtain a coating level of 6%.

### FORMULATION EXAMPLE 4

Same as for Example 1 except that 1.55 parts of the film coating solution is applied to 1 part of uncoated spheroids to obtain a coating level of 7%.

One preferred extended release formulation of this invention comprises those of the active ingredient in spheroids comprised of microcrystalline cellulose and, optionally, hydroxypropylmethylcellulose coated with a mixture of ethyl cellulose and hydroxypropyl methyl cellulose. Preferably, the spheroids are comprised of, about 30% to 40% O-desmethyl venlafaxine hydrochloride by weight, about 50% to about 70% microcrystalline cellulose, NF, by weight, and from about 0.25% to about 1% by weight of hydroxypropylmethylcellulose, USP, and coated with from about 2% to about 12% of total weight of film coating comprised of from about 80% to about 90% by weight of film coating of ethyl cellulose, NF, and from about 10% to about 20% by weight of film coating of hydroxypropylmethylcellulose, USP.

A specific extended release formulation according to the paragraph above is wherein the spheroids are composed of about 37% by weight of the O-desmethyl venlafaxine enantiomer, about 0.5% by weight of hydroxypropylmethylcellulose 0.2208, and about 62% by weight of microcrystalline cellulose. Another set of preferred compositions of this type are those wherein the film coating is comprised of ethyl cellulose (4.81% of total weight) and hydroxypropylmethylcellulose (0.85% of total weight). In another such composition the film coating comprises 6-8% by weight of total weight, such as a film coating comprised of ethyl cellulose (2.48% of total weight) and hydroxypropylmethylcellulose (0.437% of total weight).

Yet another composition according to this invention are those wherein the film coating composition is comprised of ethyl cellulose having a 44.0-51.0% content of ethoxy groups and hydroxypropylmethylcellulose having a methoxy content of 28.0-30.0% and a hydroxypropoxy group content of 7.0-12.0%. Film coating compositions of this type may be comprised of about 85% by total weight of film coating of ethyl cellulose having a 44.0-51.0% content of ethoxy groups, and about 15% by total weight of film coating of hydroxypropylmethylcellulose having a methoxy content of 28.0-30.0% and a hydroxypropoxy group content of 7.0-12.0%. A more specific film coating composition of this sort is comprised of 85% by weight of ethyl cellulose type HG 2834 and 15% by weight of hydroxypropylmethylcellulose type 2910.

Another extended release formulation for once daily administration of this invention comprises the O-desmethyl venlafaxine enantiomer, or a salt or hydrate thereof, which comprises spheroids containing 37.3% O-desmethyl venlafaxine enantiomer, 62.17% microcrystalline cellulose and 0.5% hydroxypropylmethylcellulose type 2208, coated with a quantity of a mixture comprised of 85% ethyl cellulose type HG 2834 and 15% hydroxypropylmethylcellulose type 2910 sufficient to give coated spheroids having a dissolution profile which gives the desired release rate over a 24 hour period.

A further extended release formulation of this invention is manufactured such that the spheroids are comprised of about 6% to 40% active compound by weight, about 50% to about 940% microcrystalline cellulose, NF, by weight, and, optionally, from about 0.25% to about 1% by weight of hydroxypropylmethylcellulose, USP, and coated with from about 2% to about 12% of total weight of film coating comprised of from about 80% to about 90% by weight of film coating of ethyl cellulose, NF, and from about 10% to about 20% by weight of film coating of hydroxypropylmethylcellulose, USP. A preferred subset of these extended release formulations are those wherein the spheroids are composed of about 8.25% by weight of active compound, or a pharmaceutically acceptable salt or hydrate thereof, and about 91.75% by weight of microcrystalline cellulose, with a coating of from 3 to 5% by weight of the total weight. Another preferred subset or group are those formulations wherein the spheroids are composed of about 16.5% by weight of active drug agent and about 83.5% by weight of microcrystalline cellulose, with a coating of from 4 to 6% by weight of the total weight.

In other pharmaceutical compositions and formulations of this invention, the active ingredient comprises venlafaxine hydrochloride combined with the O-desmethyl enantiomer, with the non-active ingredients being those described herein or in other formulations for venlafaxine hydrochloride known in the art.

Uses of these extended release formulations may be described as a method for providing a therapeutic blood plasma concentration of active drug compound(s) over a 24 hour period with diminished incidences of nausea and emesis which comprises administering orally to a patient in need thereof, an encapsulated, extended release formulation that provides a peak blood plasma level of active agent in from about four to about eight hours, said formulation containing O-Desmethyl venlafaxine, or a salt or salt hydrate thereof, as the active ingredient. The methods are also useful for eliminating the troughs and peaks of drug concentration in a patients blood plasma attending the therapeutic metabolism of plural daily doses of active ingredient(s) which comprises administering orally to a patient in need thereof, an encapsulated, extended release formulation that provides a peak blood plasma level of the active agent in from about four to about eight hours, said formulation containing O-Desmethyl venlafaxine, or a salt or salt hydrate thereof, as the active ingredient.

1. A composition of matter comprising R(−)-4-[2-(Dimethylamino)-1-(1-hydroxycyclohexyl)ethyl]phenol, or a pharmaceutically acceptable salt or salt hydrate thereof, substantially free of S(+)-4-[2-(Dimethylamino)-1-(1-hydroxycyclohexyl)ethyl]phenol, or a pharmaceutically acceptable salt or salt hydrate thereof.

2. A composition of matter comprising S(+)-4-[2-(Dimethylamino)-1-(1-hydroxycyclohexyl)ethyl]phenol, or a pharmaceutically acceptable salt or salt hydrate thereof, substantially free of R(−)-4-[2-(Dimethylamino-1-(1-hydroxycyclohexyl)ethyl]phenol, or a pharmaceutically acceptable salt or salt hydrate thereof.

3. A pharmaceutical composition comprising one or more pharmaceutically acceptable carriers and a pharmaceutically effective amount of R(−)-4-[2-(Dimethylamino)-1-(1-hydroxycyclohexyl)ethyl]phenol, or a pharmaceutically acceptable salt or salt hydrate thereof, substantially free of S(+)-4-[2-(Dimethylamino)-1-(1-hydroxycyclohexyl)ethyl]phenol, or a pharmaceutically acceptable salt or salt hydrate thereof.

4. A pharmaceutical composition comprising one or more pharmaceutically acceptable carriers and a pharmaceutically effective amount of S(+)-4-[2-(Dimethylamino)-1-(1-hydroxycyclohexyl)ethyl]phenol, or a pharmaceutically acceptable salt or salt hydrate thereof, substantially free of R(−)-4-[2-(Dimethylamino)-1-(1-hydroxycyclohexyl)ethyl]phenol, or a pharmaceutically acceptable salt or salt hydrate thereof.

5. A method of treatment of depression in a mammal, the method comprising administering to a mammal in need thereof a pharmaceutically effective amount of R(−)-4-[2-(Dimethylamino)-1-(1-hydroxycyclohexyl)ethyl]phenol, or a pharmaceutically acceptable salt or salt hydrate thereof, substantially free of S(+)-4-[2-(Dimethylamino)-1-(1-hydroxycyclohexyl)ethyl]phenol, or a pharmaceutically acceptable salt or salt hydrate thereof.

6. A method of treatment of depression in a mammal, the method comprising administering to a mammal in need thereof of a pharmaceutically effective amount of S(+)-4-[2-(Dimethylamino)-1-(1-hydroxycyclohexyl)ethyl]phenol, or a pharmaceutically acceptable salt or salt hydrate thereof, substantially free of R(−)-4-[2-(Dimethylamino)-1-(1-hydroxycyclohexyl)ethyl]phenol, or a pharmaceutically acceptable salt or salt hydrate thereof.
